(19) United States
(12) Patent Application Publication
(10) Pub. No.: US 2017/0258444 A1
(43) Pub. Date: Sep. 14, 2017

(54) Device for Assessment of Whole-Body Hydration Status

(71) Applicant: Artann Laboratories Inc., Trenton, NJ (US)

(72) Inventors: Armen P. Sarvazyan, Lambertville, NJ (US); Sergey Tsyuryupa, Westampton, PA (US)

(21) Appl. No.: 15/446,414

(22) Filed: Mar. 1, 2017

Related U.S. Application Data
(60) Provisional application No. 62/305,114, filed on Mar. 8, 2016.

(51) Int. Cl. A61B 8/00; A61B 8/08; A61B 5/00

(52) U.S. Cl. CPC A61B 8/4455; A61B 5/4875; A61B 8/08; A61B 8/4477; A61B 8/4411; A61B 8/48

(57) ABSTRACT

A whole-body hydration monitor detects the velocity of sound in soft tissues, which is used to determine the hydration status of a body. A set or interchangeable ultrasonic probes is provided to be easily mounted on a housing containing a controller. Each ultrasonic probe features a pair of ultrasonic transducers facing each other and spaced apart at a predetermined distance defining the acoustic base. The controller is configured to send at least one ultrasonic pulse from an emitting transducer and detect the propagation time upon arrival to the receiving transducer. Ultrasound velocity is determined using the measured propagation time and a known acoustic base of the currently connected ultrasonic probe. Magnetic connection provides for easy detachment of one probe and attachment of another with a greater or smaller acoustic base depending on the size of the muscle selected for hydration determination.

### CROSS-REFERENCE DATA

This patent application claims a priority benefit from a provisional patent application No. 62/305,114 filed Mar. 8, 2016 entitled “Method and device for assessment of whole-body hydration status”, which is incorporated herein by reference in its entirety.

### GOVERNMENT-FUNDED RESEARCH

This invention was made with government support under R44AG042990 awarded by the National Institute on Aging of the National Institutes of Health. The government has certain rights in the invention.

### BACKGROUND OF THE INVENTION

The present invention relates generally to hydration monitors. More particularly, the invention describes a C-shaped device with a pair of ultrasound transducers configured to emit and detect an ultrasound pulse propagated through tissue so as to calculate the hydration status of the tissue based on propagation speed of ultrasound signals therethrough.

Dehydration is becoming a prevalent health issue among the elderly population, especially in nursing home facilities. Approximately a third of the 3 Million elderly Americans that reside in Skilled Nursing Facilities each year suffer from dehydration during their stay. The need to maintain proper hydration levels grows as the global population ages. Dehydration is a sentinel health event among older persons in nursing homes portending serious secondary and life-threatening problems and is a prime target for litigation.

It is estimated that dehydration accounts for 10% of hospital admissions in those over age 65. Furthermore, this diagnosis has been associated with a mortality of up to 50% and recognition that identification and management of hydration status in the elderly is challenging. The etiology of the dehydration reflects both intrinsic and extrinsic factors that make this population susceptible. The intrinsic physiologic changes that accompany aging are well recognized and include: altered thirst sensation, reduction in renal function, modification of aldosterone secretion, release of vasopressin, and renin activity. In the context of increased susceptibility, extrinsic factors become critical and expand the problem of dehydration. First, significant demographic shifts and longer life expectancy expand the number of people at risk. Second, the increased use of medications by this population alters both cognitive and renal functions. Diuretics and antihypertensives alter renal blood flow and affect fluid and electrolyte absorption. Psychoactive agents used in large numbers in this population affect the ability of the elderly to respond to changes in the body fluid volume status.

The importance of dehydration is evidenced by several recent publications from different countries all identifying dehydration as a critical public health problem. A review of 35 studies in different countries on the relationship between ambient temperature and mortality concluded that elevated temperature was associated with increased risk of death from cardiovascular, respiratory, cerebrovascular, and cardiovascular causes. The elderly over age 65 were particularly vulnerable as were infants and young children. The prevailing hypothesis to explain this phenomenon is that when body temperature rises there is a concomitant shift in blood flow and overall hydration status which subsequently stress cardiopulmonary function. These patients will often present with pyrexia as their thermoregulation fails and hypernatremic or hypovolemic dehydration due to excessive water and solute loss.

Diminished thirst response occurs both in healthy adults over age 65 and those who have altered central nervous system function such as cerebrovascular accidents or Alzheimers. Physical restrictions such as blindness, arthritis, stroke also limit access to water exacerbating any underlying need to increase intake. Finally, in institutional settings fluid equilibrium can be modified by enteral and parenteral routes in quantities that may exceed the capacity to regulate balance. Thus, both under and overhydration may occur in this setting.

Renal function is critical to maintenance of water homeostasis and renal mass declines by approximately one-third by age 80 with a commensurate decrease in the number of glomeruli. The decrease in filtration area, thickening of the glomerular basement membrane, altered tubular function and changes in vascular perfusion impair the ability to excrete a water load. In addition, there is an age-related change in renal concentrating capacity. Water deprivation is accompanied by a decrease in urine flow and increase in urine osmolarity. In the elderly there is a decline in urine concentrating ability. Salt wasting is also seen in the aging kidney as evidenced by continued urinary sodium excretion in the presence of salt restriction. The significant changes in renal function increase in susceptibility to dehydration and changes in sodium balance.

The third component of total body water (TBW) regulation that undergoes significant alterations with aging is the hormonal system that alters fluid and electrolyte absorption and secretion. Studies of vasopressin, an important regulator of fluid balance, response in the elderly demonstrate altered responsiveness such that the kidney is impaired from prompt water excretion in response to excess water loads. The renin-angiotensin-aldosterone system regulates renal perfusion and salt and water absorption. Healthy older individuals have lower levels of rennin and aldosterone in response to sodium depletion than young adults.

Prevention is key and the primary way to prevent dehydration is to monitor it on a continuous basis. Availability of a portable, easy-to-use and cost-effective hydration assessment device could allow a shift from costly hospital admission to hydration monitoring and early intervention. Healthcare facilities are challenged by the lack of equipment or technology to noninvasively monitor hydration status.

### Currently Available Tools to Assess the Hydration Status

All currently available methods are not well suited for quantitative measure of body hydration changes in the institutionalized elderly (rapid, technically simple and not easily confounded). Total body water is currently assessed by the following groups of methods:

### Dilution Methods

The basic principle of the dilution techniques for body composition analysis is that the volume of a compartment can be defined as the ratio of the dose of a tracer, administered orally or intravenously, to its concentration in that body compartment within a short time after the dose is administered. Typically, two blood (or urine) samples are collected: one just before administration of the dose to determine the natural background levels and the second sample, after waiting a sufficient amount of time for penetration of the tracer within the compartment of interest. The method of analysis is dependent on the choice of tracer. For each of these tracers, the estimated error for a TBW measurement is typically up to 1 kg. In general, TBW values, obtained using the dilution technique are considered to be the reference values for comparison with alternate measurement techniques. However, the dilution methods are impractical because of their high costs, need of special facilities and trained personnel.

### Bioelectrical Impedance and Conductance Methods

The aqueous tissues of the body, due to their dissolved electrolytes, are the major conductors of an electrical current, whereas body fat and bone have relatively poor conductance properties. Although significant technical problems eliminated the viability of many electrical methods for in vivo body composition analyses, the basic principle of measuring TBW has been suggested and several commercial instruments designed for bioelectrical impedance analysis (BIA) were marketed. At present, despite its limited accuracy, it is probably the most frequently used method, due mainly to the relatively inexpensive cost of the basic instrument, its ease of operation, and its portability.

Oversimplifications in formulae in the standard BIA methods lead to problems. A more complex model is based on modification of a mixture theory model and partitioning the whole body into segments modeled as cylinders measuring resistance and reactance over a wide range of frequencies. The technique based on this model is called bioelectrical impedance spectroscopy (BIS) and a commercially available instrument is known (Xitron, San Diego, Calif.).

Regardless of the choice of single- or multi-frequency method, many investigators found that the basic model failed; that is, the impedance index alone was not an accurate predictor and that additional anthropometric terms (i.e., weight, age, gender, race, shoulder width, girth, waist-to-hip ratio and body mass index) were included in the prediction model to reduce the standard error of the estimate. Clinical studies demonstrated that in assessing geriatric in-patients, there is little concordance between the clinical and the bioelectrical evaluation of the hydration status. In studies, concordance between the results of clinical judgment and bioelectrical impedance measurements was only 43.7%.

An alternate bioelectrical method used to measure body composition is total body electrical conductivity (TOBEC). It is based on the premise that, when a body is placed inside a solenoid generating a time-varying electromagnetic field, eddy currents are induced in the conductive tissues in the body. Two commercial instruments were developed (EM-Scan, Springfield, Ill.), one sized for infants and the other for adults. The basic TOBEC concept would indicate that it is relatively insensitive to shifts of fluid or electrolyte between the intracellular and extracellular compartments; hence, it has only been used to monitor TBW. However, it was suggested that using multifrequency TOBEC coupled with Fourier analysis might provide a measure of each fluid subcompartment, although no subsequent studies have demonstrated this application.

### Magnetic Resonance Imaging (MRI)

Muscle provides the largest body reservoir for water and its volume decreases with dehydration. MRI has been used for assessment of muscle volume and composition but uses bulky stationary equipment and thus is impractical for field TBW measurements.

### Hydration Markers of Plasma and Urine

Plasma and urine osmolality provides the best available hydration markers for hyperosmotic-hypovolemic dehydration in a laboratory setting. However, urine osmolality may reflect the recent volume of fluid consumed rather than the overall state of hydration. The intake of a large volume of water rapidly dilutes the plasma and the kidneys excrete diluted urine even if dehydration exists. With these potential limitations, plasma and urine osmolality in combination are considered valid laboratory methods for the assessment of hydration status.

In summary, to date none of the known techniques for dehydration assessment have achieved widespread success because of their cost, complexity, lack of portability, invasiveness or lack of predictive value. The at-risk population is expanding as resources are contracting. Furthermore, the morbidity of dehydration that is not recognized or undertreated is likely to be significant. There is a significant need for a sensitive, compact and non-invasive device for the assessment of hydration status in elderly as well as in infants, which requires no special education for personnel and allows for repeated testing with little clinician effort and patient risk.

### SUMMARY OF THE INVENTION

Accordingly, it is an object of the present invention to overcome these and other drawbacks of the prior art by providing a novel hydration monitor device which is simple to use and not costly to manufacture.

It is another object of the present invention to provide a hydration monitoring device with easily interchangeable ultrasonic probes, which contain ultrasound elements of the device.

The general concept of using ultrasound waves for measuring hydration status of the tissue has been described in greater detail in our previous patents, see U.S. Pat. No. 7,033,321 and U.S. Pat. No. 7,291,109, incorporated herein by reference in their respective entireties. This concept is based on assessment of the body hydration status using the measurement of speed of ultrasound propagation in muscle, which is the largest body reservoir for water and so muscle water content (MWC) closely reflects overall hydration status of the body. Although MWC is informative on the overall body hydration status, none of the conventional methods are based on the measurements of MWC as it is realized in the proposed ultrasonic method. Similar to MRI, ultrasound velocity is equally sensitive to both intracellular and extracellular water. Physical foundation of the ultrasonic method of tissue water content assessment is presented in the review paper on acoustic properties of soft biological tissues [Sarvazyan A P, Lyrchikov A G. Correlation of bulk elastic properties of soft biological tissues with content of water, protein and fat. Biomechanics in Medicine and Surgery, Riga, 1986; 1: 353-358.], which is incorporated herein by reference. Briefly stated, velocity of ultrasonic waves propagation depends on bulk compressibility and density of medium and both these parameters are defined by short range molecular interactions. Water is a major molecular component of tissue, therefore interactions of water molecules with all organic and inorganic molecules are defining the value of ultrasound velocity in tissue and changes in the water content result in changes in the ultrasound velocity in tissue. It was shown that the ultrasound velocity in soft tissues is a linear function of water content. In muscle tissue, the slope of the velocity versus the water content is about 3.5 m/s per 1% change in water content.

As compared with prior art devices, the present invention features a plurality of easily interchangeable C-shaped ultrasonic probes, which contain a pair of facing each other emitting and receiving ultrasound transducers located at the opposite ends of the probe. The housing of the device contains a controller operably connected to the presently attached ultrasonic probe and configured to provide a triggered generation of excitation ultrasound pulses. The emitting transducer is used to emit the triggering ultrasonic pulse towards the receiving transducer so as to measure the time of ultrasound propagation between the transducers.

The C-shaped ultrasonic probes are provided in a range of suitable sizes, each defining the unique distance between the ultrasonic transducers, defining its acoustic base. The probes are configured to be easily removed and re-attached to the housing using a quick-connect coupling, such as a magnetic coupling.

In use, the controller is configured to measure the propagation time of ultrasound and using a fixed acoustic base of the presently attached probe calculate the ultrasound velocity through tissue. This is used to determine the hydration status of the muscle and correspondingly the hydration status of the body.

The use of a plurality of interchangeable C-shaped ultrasonic probes greatly simplifies both mechanical and electronic parts of the device. In addition, it simplifies the measuring procedure, improves its reproducibility by eliminating an error of the distance measurement and greatly decreases the cost of the device.

### BRIEF DESCRIPTION OF THE DRAWINGS

Subject matter is particularly pointed out and distinctly claimed in the concluding portion of the specification. The foregoing and other features of the present disclosure will become more fully apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings, in which:

FIG. 1 is a general view of the body hydration monitor in use;

FIG. 2 is a first perspective view of the body hydration monitor;

FIG. 3 is a second perspective view thereof;

FIG. 4 is a first perspective view of the body hydration monitor controller;

FIG. 5 is a second perspective view thereof;

FIG. 6 is a top view of the body hydration monitor controller;

FIG. 7 is a rear view of the probe;

FIG. 8 is a first perspective view of the probe;

FIG. 9 is a second perspective view thereof;

FIG. 10 is a schematic block-diagram of the probe; and

FIG. 11 is a schematic block-diagram of the controller.

### DETAILED DESCRIPTION OF THE PREFERRED EMBODIMENT OF THE INVENTION

The following description sets forth various examples along with specific details to provide a thorough understanding of claimed subject matter. It will be understood by those skilled in the art, however, that claimed subject matter may be practiced without one or more of the specific details disclosed herein. Further, in some circumstances, well-known methods, procedures, systems, components and/or circuits have not been described in detail in order to avoid unnecessarily obscuring claimed subject matter. In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here. It will be readily understood that the aspects of the present disclosure, as generally described herein, and illustrated in the figures, can be arranged, substituted, combined, and designed in a wide variety of different configurations, all of which are explicitly contemplated and make part of this disclosure.

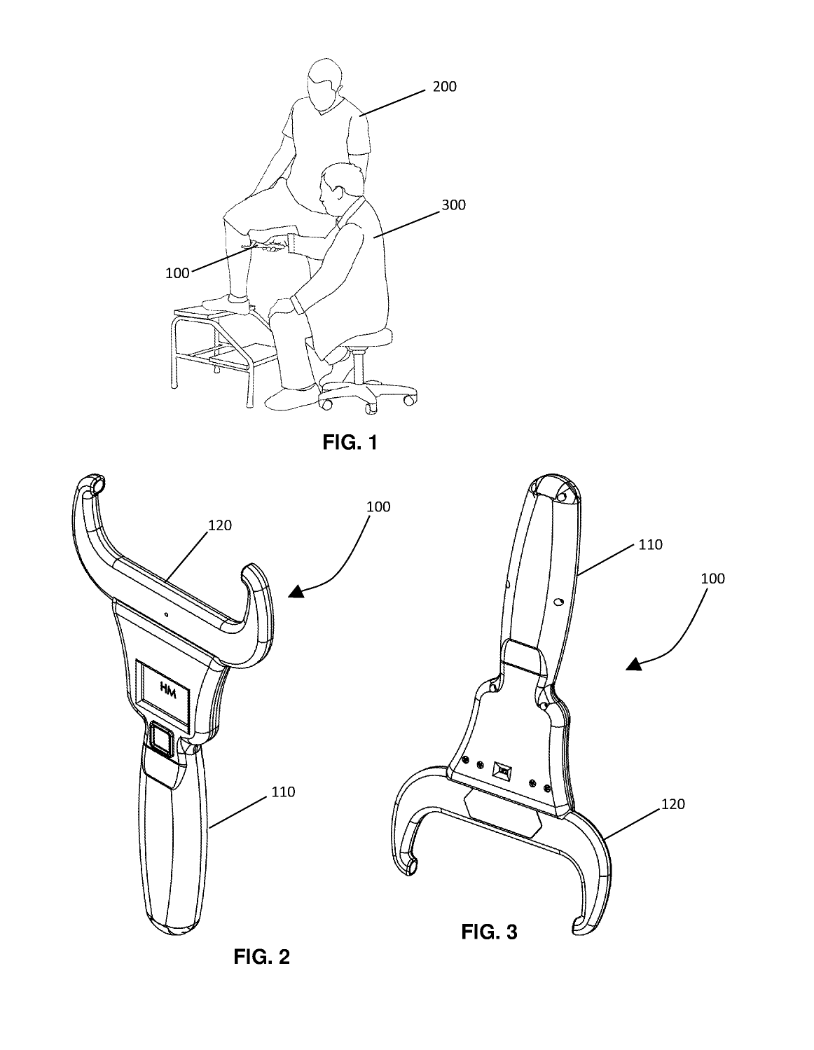

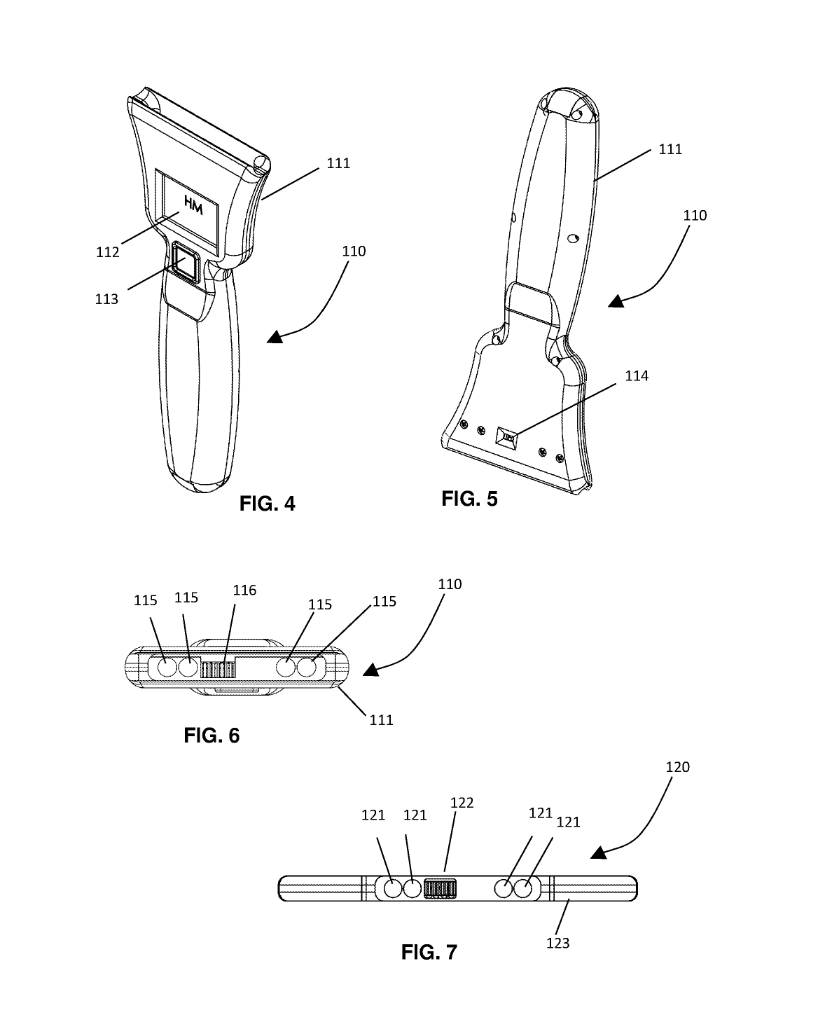

The Body Hydration Monitor (HM) is an ultrasonic device for monitoring the hydration status of a human body by measuring the velocity of sound propagation in a muscle. FIG. 1 shows the application of the HM device 100 by an operator 300 to evaluate the hydration status of a subject 200. The device 100 includes of a controller 110 in a housing from which the device may be operated and set of interchangeable ultrasonic probes 120 (FIG. 2 and FIG. 3). The device 100 may be configured to detect the velocity of sound in soft tissues, which can in turn be used to calculate its water content to determine the hydration status of a body. The “pitch-catch” method may be used for ultrasonic velocity measurement. The method may be based on determination of ultrasonic pulse arrival time with respect to pulse emitting time.

Each ultrasonic probe 120 may be characterized by a fixed and predetermined distance between a transmitting transducer and a receiving transducer defining its respective acoustic base. In embodiments, a plurality of probes may be provided with respective acoustic base distance suitable to fit over a corresponding muscle selected for measuring. FIG. 1 shows the device 100 applied on subject's 200 soleus muscle; however another muscle can be used for measurements as well, such as the biceps.

The ultrasound velocity may be calculated from the measured value of the ultrasonic pulse propagation time or time-of-flight, based on the device calibration data, which may be stored in the memory of the controller.

An LCD or another type of a display 112 may be used to monitor device operation and displaying the measurement results. The device operation may be controlled by multifunctional buttons 113, such as a button allowing left-right and up-down movements. The device may also include a power switch 114. The housing of the controller may be shaped into a handle and may have a specific arrangement for mounting the ultrasonic probe 120 that is shown in detail in FIG. 6.

A quick-connect coupling may be provided to allow rapid attachment and detachment of individual ultrasonic probes 120 to and from the housing of the controller 110. In embodiments, the coupling may include:
a retention portion configured for attachment and detachment of one of the ultrasonic probes 120 at a time to and from the housing, and
an electrical connection portion including a first plurality of electrical contacts 116 located on the housing and a corresponding second plurality of electrical contacts 122 located on the ultrasonic probe 120

The electrical contacts may be designed in such a way that when the ultrasonic probe 120 is attached to the housing, the first plurality of electrical contacts 116 are operably coupled with the second plurality of electrical contacts 120. This will complete an electrical circuit for the controller 110 to operate the ultrasonic transducers 125 and 126 on the ultrasonic probe 120 as described herein.

The retention portion of the quick-connect coupling may utilize a variety of quick-connect mechanical engagement features, such as a sliding design, a snap-on design, a bayonet ¼ turn attachment design, etc. In one exemplary embodiment, two or four pairs of magnets may be used to provide secure connection of the ultrasonic probe 120 and the housing of the controller 110. In this case, housing magnets 115 may be located on the coupling portion of the housing and a corresponding set of magnets 121 may be located on the ultrasonic probe 120. To assure proper connection between corresponding electrical contacts 116 and 122, the polarity of magnets 115 and 121 may be selected to avoid improper orientation of the ultrasonic probe 120 relative to the housing of the controller 110. This approach may be used to exclude a possibility of reverse attachment. The spring-loaded contacts of a modular connector 116 may be configured to mate with the corresponding flat contacts of connector 122 of the ultrasonic probe 120 (see FIG. 7).

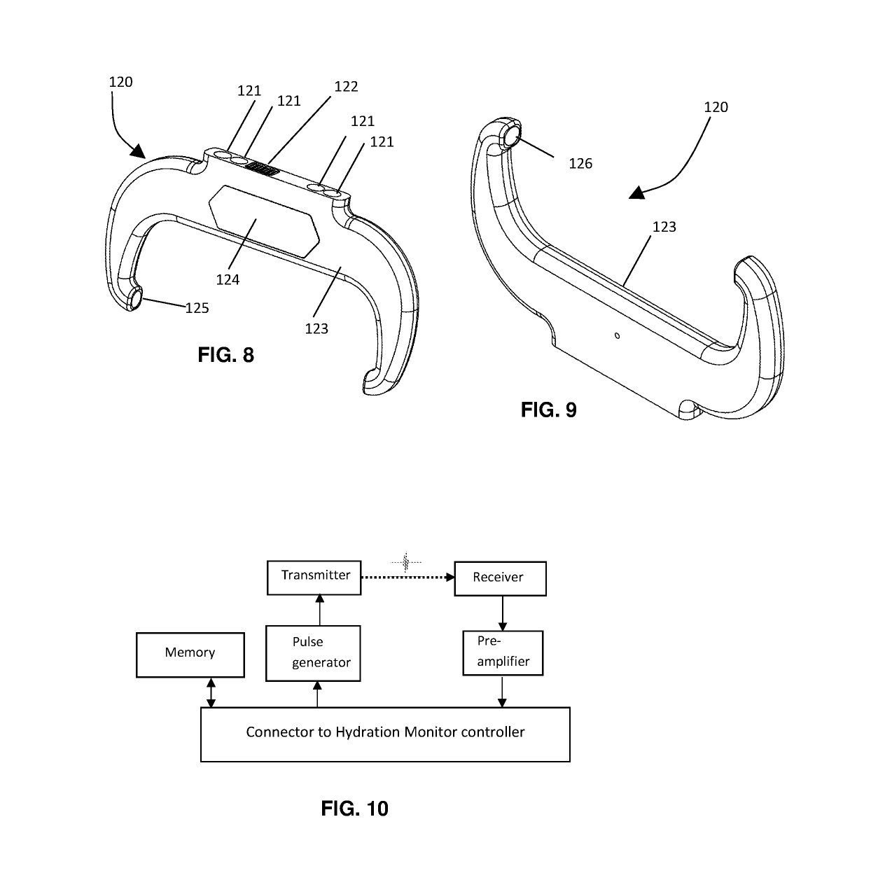

A plurality of ultrasonic probes 120 may be provided to mate with the housing of the controller 110. Each detachable ultrasonic probe 120 (shown in perspective view in FIG. 8 and FIG. 9) may be made as a C-shape body 123 with a short universal tail feature of the quick connect coupling that mates to the corresponding slot of the housing of the controller 110. The spaced apart ends or tips of the probe 120 may be formed to house a pair of the ultrasonic transducers 125 and 126. Each ultrasonic probe 120 may be designed to have a specific and predetermined distance between transducers 125 and 126, known as the acoustic base. A set of ultrasonic probes 120 with different size acoustic bases may be provided so as to cover the suitable range of dimensions of potential sites of application on a human body. Anywhere between 3 and 6 probes may be provided. Each probe having the same design as the next but with a different acoustic base. In one example, a set of four probes for adult use may be provided with the acoustic base set at 60 mm, 80 mm, 100 mm, and 120 mm. Suitable plurality of probes may also be produced for a pediatric and even a neonate applications.

One transducer 125 (see FIG. 8) of the ultrasonic probe 120 may be used as a transmitter while the other transducer 126 (see FIG. 9) may be used as a receiver. Both transducers 125 and 126 may be identical in construction and consist of one or more piezoceramic disks mounted in the tip edge and facing each other. The piezoceramic disk may have a bull's-eye electrode pattern that provides easy assembly for the electrical connection of the external electrode to the probe body 123.

The ultrasonic probe electronics may be placed in the interior of the C-shaped body and may be protected by a cover 124. The ultrasonic probe body 123 and cover 124 may be made from metal and with external metalized electrode of transducers 125 and 126 provide efficient shielding to protect received signal from electromagnetic interference (noise). The probe design may be made without any cavities to improve the ease of cleaning and disinfection.

A block-diagram of the electronic module of the ultrasonic probe 120 is shown in FIG. 10. The ultrasonic probe 120 may be configured to generate a burst ultrasonic pulse in response to a triggering pulse from HM controller 110. In one example, HM 100 may emit short ultrasound pulses with frequency in the range of about 0.5 to about 5 MHz. These ultrasound pulses may be emitted by the transducer 125. Ultrasound signal then propagates through the muscle tissue and the response of the receiving transducer 126 may be detected and amplified and then sent back to the controller 110 for data processing. The probe's specific technical information (calibration, acoustic base, etc.) may be saved in the embedded EEPROM chip or another computer memory as appropriate. The data stored in the probe memory may be read by the controller 110 when electrical contacts 116 and operably connected to the corresponding electrical contacts 122 of the presently attached probe 120. In embodiments, ultrasonic probes 120 may be interchanged without any need to re-program or re-calibrate the device 100.

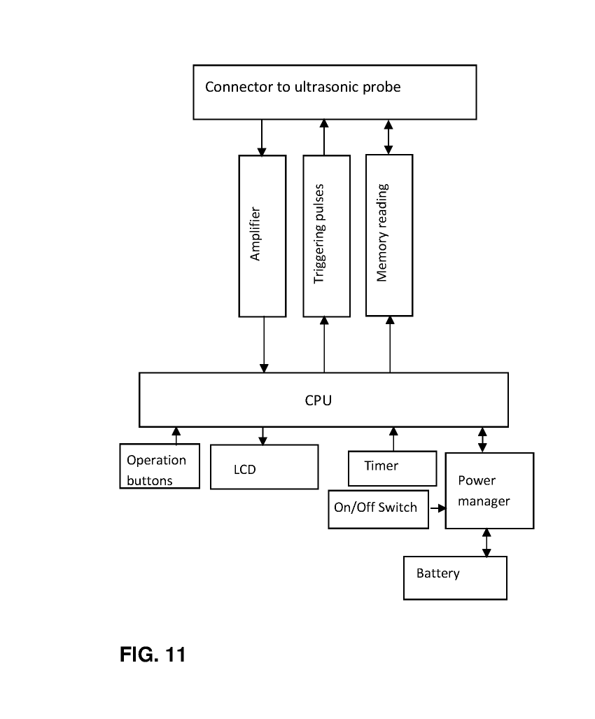

The block diagram of the electronic module of the controller 110 is shown in FIG. 11. A rechargeable battery may be used to provide device power. The multifunctional buttons 113 or another suitable user interface arrangement may be used to provide user control of the device 100. Current device status and results of hydration measurements may be shown on an LCD or another suitable display. In alternative embodiments, a simple light indicator may also be used such as GREEN for adequate hydration and RED for dehydration status. The CPU may be used to control device functionality in accordance with the operation algorithm. The controller 110 may be further configured to automatically recognize a specific acoustic base dimension of the presently connected ultrasonic probe 120 by reading the probe's memory, so that the device of the invention may be ready for measurements right after device assembly (installation or a desirable ultrasonic probe 120 on the housing of the controller 110).

When START button is pressed, the controller 110 may be configured to send to the ultrasonic probe 120 a triggering pulse to cause an ultrasound pulse to be sent from the ultrasound transducer 125 towards the ultrasound transducer 126. The controller 110 may amplify the received signal and measure the ultrasound pulse propagation time. The arrival point may be determined as intersection of receiving signal with zero level after the first half-wave. Using this measurement, the controller 110 may calculate ultrasound velocity based on predetermined calibration data and a specific acoustic base of an ultrasonic probe 120 presently connected to the controller 110.

The calibration procedure conducted during device manufacturing or repeated prior to use and may take into account the ultrasonic probe's 120 and controller's 110 specific parameters (probe's base, time correction constant, etc.) that are saved in the ultrasonic probe's 120 memory. The device calibration may be made using NaCl aqueous solution with known ultrasound velocity. The CPU of the controller 110 may provide accurate measurement of the pulse time-of-flight. The ultrasound velocity measurements results may be stored in the controller's 110 memory and can be uploaded to a PC for future analysis.

The HM controller 110 may be designed for self-sufficient device operation and keeping in memory measurements for multiple subjects. The HM controller 110 may further contain a power manager chip to provide the battery recharging. The power switch (On/Off) may be used to save power while the device is in storage.

To further increase device accuracy, various embodiments of the device 100 may be programmed to send not one but a predetermined plurality of ultrasonic pulses and calculate average pulse propagation time.

It is contemplated that any embodiment discussed in this specification can be implemented with respect to any method of the invention, and vice versa. It will be also understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, Aft AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, Aft BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12, 15, 20 or 25%.

All of the devices and/or methods disclosed and claimed herein can be made and executed without undue experimentation in light of the present disclosure. While the devices and methods of this invention have been described in terms of preferred embodiments, it will be apparent to those of skill in the art that variations may be applied to the devices and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

What is claimed is:

1. A device for assessment of whole-body hydration status, said device comprising:
a plurality of interchangeable C-shaped ultrasonic probes, each ultrasonic probe containing a pair of ultrasonic transducers facing each other and located on opposite ends of said ultrasonic probe at a predetermined fixed distance defining an acoustic base, said acoustic base is selected to be different for each ultrasonic probe,
a housing containing a controller and a user interface, said controller configured to operate said pair of ultrasonic transducers of the currently attached ultrasonic probe by causing a first ultrasonic transducer to emit an ultrasonic pulse towards a second ultrasonic transducer, said controller is further configured to determine propagation time of said ultrasonic pulse between said ultrasonic transducers and determine whole-body hydration status based on said propagation time and said acoustic base for the currently attached ultrasonic probe,
wherein each of said ultrasonic probes is configured to be detachably attached to said housing via a quick-connect coupling comprising:
a retention portion configured for attachment and detachment of one of said ultrasonic probes at a time to and from said housing, and
an electrical connection portion comprising a first plurality of electrical contacts located on said housing and a corresponding second plurality of electrical contacts located on said ultrasonic probe, whereby when said ultrasonic probe is attached to said housing said first plurality of electrical contacts are operably coupled with said second plurality of electrical contacts so as to complete an electrical circuit for said controller to operate said ultrasonic transducers on said ultrasonic probe.

2. The device as in claim 1, wherein said retention portion comprises one or more of pairs of magnets with first magnets located on said housing and corresponding second magnets located on said ultrasonic probe.

3. The device as in claim 1, wherein said plurality of ultrasonic probes comprise a set of four probes with acoustic base at 60 mm, 80 mm, 100 mm, and 120 mm.
